SEQUENCE LISTING (88 sequences)

SEQ ID NO: 1 - protein, 4 aa, Human
NSIC

SEQ ID NO: 2 - protein, 5 aa, Human
NNSIC

SEQ ID NO: 3 - protein, 6 aa, Human
QNNSIC

SEQ ID NO: 4 - protein, 7 aa, Human
PQNNSIC

SEQ ID NO: 5 - protein, 8 aa, Human
HPQNNSIC

SEQ ID NO: 6 - protein, 9 aa, Human
IHPQNNSIC

SEQ ID NO: 7 - protein, 10 aa, Human
YIHPQNNSIC

SEQ ID NO: 8 - protein, 11 aa, Human
KYIHPQNNSIC

SEQ ID NO: 9 - protein, 12 aa, Human
GKYIHPQNNSIC

SEQ ID NO: 10 - protein, 13 aa, Human
QGKYIHPQNNSIC

SEQ ID NO: 11 - protein, 14 aa, Human
PQGKYIHPQNNSIC

SEQ ID NO: 12 - protein, 15 aa, Human
CPQGKYIHPQNNSIC

SEQ ID NO: 13 - protein, 16 aa, Human
VCPQGKYIHPQNNSIC

SEQ ID NO: 14 - protein, 17 aa, Human
SVCPQGKYIHPQNNSIC

SEQ ID NO: 15 - protein, 18 aa, Human
DSVCPQGKYIHPQNNSIC

SEQ ID NO: 16 - protein, 4 aa, Human
FCCS

SEQ ID NO: 17 - protein, 5 aa, Human
FCCSL

SEQ ID NO: 18 - protein, 6 aa, Human
FCCSLC

SEQ ID NO: 19 - protein, 7 aa, Human
FCCSLCL

SEQ ID NO: 20 - protein, 4 aa, Human
AQMC

SEQ ID NO: 21 - protein, 5 aa, Human
TAQMC

SEQ ID NO: 22 - protein, 6 aa, Human
QTAQMC

SEQ ID NO: 23 - protein, 7 aa, Human
DQTAQMC

SEQ ID NO: 24 - protein, 8 aa, Human
YDQTAQMC

SEQ ID NO: 25 - protein, 9 aa, Human
YYDQTAQMC

SEQ ID NO: 26 - protein, 10 aa, Human
EYYDQTAQMC

SEQ ID NO: 27 - protein, 11 aa, Human
REYYDQTAQMC

SEQ ID NO: 28 - protein, 12 aa, Human
LREYYDQTAQMC

SEQ ID NO: 29 - protein, 13 aa, Human
RLREYYDQTAQMC

SEQ ID NO: 30 - protein, 14 aa, Human
CRLREYYDQTAQMC

SEQ ID NO: 31 - protein, 15 aa, Human
TCRLREYYDQTAQMC

SEQ ID NO: 32 - protein, 16 aa, Human
STCRLREYYDQTAQMC

SEQ ID NO: 33 - protein, 17 aa, Human
GSTCRLREYYDQTAQMC

SEQ ID NO: 34 - protein, 18 aa, Human
PGSTCRLREYYDQTAQMC

SEQ ID NO: 35 - protein, 19 aa, Human
EPGSTCRLREYYDQTAQMC

SEQ ID NO: 36 - protein, 20 aa, Human
PEPGSTCRLREYYDQTAQMC

SEQ ID NO: 37 - protein, 21 aa, Human
APEPGSTCRLREYYDQTAQMC

SEQ ID NO: 38 - protein, 22 aa, Human
YAPEPGSTCRLREYYDQTAQMC

SEQ ID NO: 39 - protein, 23 aa, Human
PYAPEPGSTCRLREYYDQTAQMC

SEQ ID NO: 40 - protein, 24 aa, Human
TPYAPEPGSTCRLREYYDQTAQMC

SEQ ID NO: 41 - protein, 25 aa, Human
FTPYAPEPGSTCRLREYYDQTAQMC

SEQ ID NO: 42 - protein, 26 aa, Human
AFTPYAPEPGSTCRLREYYDQTAQMC

SEQ ID NO: 43 - protein, 27 aa, Human
VAFTPYAPEPGSTCRLREYYDQTAQMC

SEQ ID NO: 44 - protein, 28 aa, Human
QVAFTPYAPEPGSTCRLREYYDQTAQMC

SEQ ID NO: 45 - protein, 29 aa, Human
AQVAFTPYAPEPGSTCRLREYYDQTAQMC

SEQ ID NO: 46 - protein, 30 aa, Human
PAQVAFTPYAPEPGSTCRLREYYDQTAQMC

SEQ ID NO: 47 - protein, 31 aa, Human
LPAQVAFTPYAPEPGSTCRLREYYDQTAQMC

SEQ ID NO: 48 - protein, 4 aa, Human
RLCA

SEQ ID NO: 49 - protein, 5 aa, Human
RLCAP

SEQ ID NO: 50 - protein, 6 aa, Human
RLCAPL

SEQ ID NO: 51 - protein, 7 aa, Human
RLCAPLR

SEQ ID NO: 52 - protein, 8 aa, Human
RLCAPLRK

SEQ ID NO: 53 - protein, 9 aa, Human
RLCAPLRKC

SEQ ID NO: 54 - protein, 10 aa, Human
RLCAPLRKCR

SEQ ID NO: 55 - DNA, 32 bp, Artificial Sequence
  Description of Artificial SequenceSynthetic
cgctctagaccaccatgggcctctccaccgtg

SEQ ID NO: 56 - protein, 6 aa, Artificial Sequence
  Description of Artificial SequenceSynthetic
MGLSTV

SEQ ID NO: 57 - DNA, 41 bp, Artificial Sequence
  Description of Artificial SequenceSynthetic
acacagggtaacatctataccggtggtgcctgagtcctcag

SEQ ID NO: 58 - DNA, 40 bp, Artificial Sequence
  Description of Artificial SequenceSynthetic
ctgaggactcaggcaccaccggtatagatgttaccctgtg

SEQ ID NO: 59 - protein, 12 aa, Artificial Sequence
  Description of Artificial SequenceSynthetic
EDSGTTGIDVTL

SEQ ID NO: 60 - DNA, 40 bp, Artificial Sequence
  Description of Artificial SequenceSynthetic
cctctgtcgactattataagcagcttattttcacggattg

SEQ ID NO: 61 - DNA, 29 bp, Artificial Sequence
  Description of Artificial SequenceSynthetic
tcaaccggtaaatgtggaatagatgttac

SEQ ID NO: 62 - protein, 7 aa, Artificial Sequence
  Description of Artificial SequenceSynthetic
KCGIDVT

SEQ ID NO: 63 - DNA, 28 bp, Artificial Sequence
  Description of Artificial SequenceSynthetic
gtttaccggtcctaactggcttagtgtc

SEQ ID NO: 64 - protein, 6 aa, Artificial Sequence
  Description of Artificial SequenceSynthetic
PNWLSV

SEQ ID NO: 65 - DNA, 27 bp, Artificial Sequence
  Description of Artificial SequenceSynthetic
agcaccggtgaacagactttccagctg

SEQ ID NO: 66 - protein, 6 aa, Artificial Sequence
  Description of Artificial SequenceSynthetic
EQTFQL

SEQ ID NO: 67 - DNA, 27 bp, Artificial Sequence
  Description of Artificial SequenceSynthetic
ggaaaccggtccgggaaagaaagtggg

SEQ ID NO: 68 - protein, 6 aa, Artificial Sequence
  Description of Artificial SequenceSynthetic
PGKKVG

SEQ ID NO: 69 - protein, 208 aa, Rattus rattus
NCGIDVTLCEEAFFRFAVPTKIIPNWLSVLVDSLPGTKVNAESVERIKRRHSSQEQTFQLLKLWKHQNRDQEMVKKIIQDIDLCESSVQRHIGHANLTTEQLRILMESLPGKKISPDEIERTRKTCKPSEQLLKLLSLWRIKNGDQDTLKGLMYALKHLKAYHFPKTVTHSLRKTIRFLHSFTMYRLYQKLFLEMIGNQVQSVKISCL

SEQ ID NO: 70 - protein, 208 aa, Mus musculus
KCGIDVTLCEEAFFRFAVPTKIIPNWLSVLVDSLPGTKVNAESVERIKRRHSSQEQTFQLLKLWKHQNRDQEMVKKIIQDIDLCESSVQRHLGHSNLTTEQLLALMESLPGKKISPEEIERTRKTCKSSEQLLKLLSLWRIKNGDQDTLKGLMYALKHLKTSHFPKTVTHSLRKTMRFLHSFTMYRLYQKLFLEMIGNQVQSVKISCL

SEQ ID NO: 71 - protein, 208 aa, Human
KCGIDVTLCEEAFFRFAVPTKFTPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCL

SEQ ID NO: 72 - DNA, 483 bp, Human
  CDS (1)..(483)
gatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccccagattgagaat

SEQ ID NO: 73 - protein, 161 aa, Human
DSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIEN

SEQ ID NO: 74 - DNA, 705 bp, Human
  CDS (1)..(705)
ttgcccgcccaggtggcatttacaccctacgccccggagcccgggagcacatgccggctcagagaatactatgaccagacagctcagatgtgctgcagcaagtgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcagggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcactggcgac

SEQ ID NO: 75 - protein, 235 aa, Human
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASRDAVCTSTSPTRSMAPGAVHLPQPVSTRSQHTQPTPEPSTAPSTSFLLPMGPSPPAEGSTGD

SEQ ID NO: 76 - protein, 420 aa, Human
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTGKCGIDVTLCEEAFFRFAVPTKFTPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCL

SEQ ID NO: 77 - protein, 211 aa, Human
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTT

SEQ ID NO: 78 - protein, 417 aa, Human
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTGIDVTLCEEAFFRFAVPTKFTPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCL

SEQ ID NO: 79 - protein, 397 aa, Human
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTGPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCL

SEQ ID NO: 80 - protein, 366 aa, Human
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTGEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCL

SEQ ID NO: 81 - protein, 311 aa, Human
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTGPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCL

SEQ ID NO: 82 - protein, 106 aa, Human
MDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFC

SEQ ID NO: 83 - protein, 109 aa, Human
MDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSL

SEQ ID NO: 84 - protein, 109 aa, Human; identical to SEQ ID NO: 83

SEQ ID NO: 85 - protein, 101 aa, Human
MYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSL

SEQ ID NO: 86 - protein, 91 aa, Human
MCTKCHKGTYLYNDCPGPGQDTDCRECESGSFTASENHLRHCLSCSKCRKEMGQVEISSCTVDRDTVCGCRKNQYRHYWSENLFQCFNCSL

SEQ ID NO: 87 - protein, 94 aa, Human
MSISCTKCHKGTYLYNDCPGPGQDTDCRECESGSFTASENHLRHCLSCSKCRKEMGQVEISSCTVDRDTVCGCRKNQYRHYWSENLFQCFNCSL

SEQ ID NO: 88 - protein, 10 aa, Artificial Sequence
  Linker or spacer
VKGTEDSGTT

(19) United States
(12) Patent Application Publication
(10) Pub. No.: US 2003/0187224 A1
(43) Pub. Date: Oct. 2, 2003

(54) Chimeric OPG polypeptides

(75) Inventors: William J. Boyle, Moorpark, CA (US); Scott Wooden, Thousand Oaks, CA (US)

Correspondence Address: FINNEGAN, HENDERSON, FARABOW, GARRETT & DUNNER, LLP, 1300 I STREET, NW, WASHINGTON, DC, 20005, US

(73) Assignee: Amgen Inc.

(21) Appl. No.: 10/436,826

(22) Filed: May 12, 2003

Related U.S. Application Data
(63) Continuation of application No. 09/268,626, filed on Mar. 15, 1999.
Application No. 09/268,626 is a continuation of application No. 08/850,188, filed on May 1, 1997, now abandoned.

(51) Int. Cl. C07K 14/705; C07K 14/715; C12P 21/02; C12N 5/06

(52) U.S. Cl. 530/350; 530/387.3; 536/23.5; 435/69.1; 435/320.1; 435/325

(57) ABSTRACT

Chimeric polypeptides comprising fusions of an osteoprotegerin dimerization domain to a heterologous sequence are provided. Also provided are nucleic acids encoding the polypeptides, expression vectors and host cells for their production and pharmaceutical compositions comprising the polypeptides.

### FIELD OF THE INVENTION

The invention relates generally to chimeric polypeptides. More particularly, the invention relates to chimeric polypeptides comprising a fusion of an osteoprotegerin dimerization domain to a heterologous sequence. The polypeptides may be used in a variety of diagnostic and therapeutic applications.

### BACKGROUND OF THE INVENTION

Cells recognize a variety of signals which modulate growth, differentiation and metabolism. Effectors of cellular functions include small molecular weight organic compounds, carbohydrates, amino acids, peptides and proteins. At present, the best understood signalling process employs secretion of a signalling molecule from one cell to modulate functions of other cells (autocrine regulation). It has also been observed that secreted signalling molecules may also modulate the functions of cells which secrete them (paracrine regulation). The ability of cells to respond to external signals usually requires that the appropriate receptors which bind the signalling molecules be present on the cell surface. Protein-mediated signalling between cells involves binding of growth factors, hormones, cytokines, cell adhesion proteins and the like to cell surface receptors.

As a class of proteins, receptors vary in their structure and mode of signal transduction. They are characterized by having an extracellular domain that is involved in binding a signalling molecule and cytoplasmic domain which transmits an appropriate intracellular signal. Receptor expression patterns ultimately determine which cells will respond to a given ligand, while the structure of a given receptor dictates the cellular response induced by ligand binding. Receptors have been shown to transmit intracellular signals via their cytoplasmic domains by activating protein tyrosine, or protein serine/threonine phosphorylation (e.g., platelet derived growth factor receptor (PDGFR) or transforming growth factor-β receptor-I (TGFβR-I), by stimulating G-protein activation (e.g., β-adrenergic receptor), and by modulating associations with cytoplasmic signal transducing proteins (e.g., TNFR-1 and Fas/APO) (Heldin, Cell 80, 213-223 (1995)).

The tumor necrosis factor receptor (TNFR) superfamily is a group of type I transmembrane proteins which share a conserved cysteine-rich motif which is repeated three to six times in the extracellular domain (Smith, et al. Cell 76, 953-962 (1994)). Collectively, these repeat units form the ligand binding domains of these receptors (Chen et al., Chemistry 270, 2874-2878 (1995)). The ligands for these receptors are a structurally related group of proteins homologous to TNFα. (Goeddel et al. Cold Spring Harbor Symp. Quart. Biol. 51, 597-609 (1986); Nagata et al. Science 267, 1449-1456 (1995)). TNFα binds to distinct, but closely related receptors, TNFR-1 and TNFR-2. TNFα produces a variety of biological responses in receptor bearing cells, including, proliferation, differentiation, and cytotoxicity and apoptosis (Beutler et al. Ann. Rev. Biochem. 57, 505-518 (1988)).

TNFα is believed to mediate acute and chronic inflammatory responses (Beutler et al. ibid). Systemic delivery of TNFα induces septic shock-like syndrome and widespread tissue necrosis. Because of this, TNFα may be responsible for the severe morbidity and mortality associated with a variety of infectious diseases, including sepsis. Mutations in FasL, the ligand for the TNFR-related receptor Fas/APO (Suda et al. Cell 75, 1169-1178 (1993)), is associated with autoimmunity (Fisher et al. Cell 81, 935-946 (1995)), while overproduction of FasL may be implicated in drug-induced hepatitis. Thus, ligands to the various TNFR-related proteins often mediate the serious effects of many disease states, which suggests that agents that neutralize the activity of these ligands would have therapeutic value.

Soluble TNFR-1 receptors and antibodies that bind TNFα have been tested for their ability to neutralize systemic TNFFa (Loetscher et al. Cancer Cells 3, 221-226 (1991)). A naturally occuring form of a secreted TNFR-1 and TNFR-2 mRNA was recently cloned, and its product tested for its ability to neutralize TNFα activity in vitro and in vivo (Kohno et al. Proc. Natl. Acad. Sci. USA 87, 8331-8335 (1990)). The ability of this protein to neutralize TNFα suggests that soluble TNF receptors function to bind and clear TNF thereby blocking the cytotoxic effects on TNFR-bearing cells.

Recombinantly-produced TNF inhibitors have also been taught in the art. For example, EP 393 438 and EP 422 339 teach the amino acid and nucleic acid sequences of a “30 kDa TNF inhibitor” (also known as a p55 receptor) and a “40 kDa inhibitor” (also known as a p75 receptor) as well as modified forms thereof, e.g., fragments, functional derivatives and variants. EP 393 438 and EP 422 339 also disclose methods for isolating the genes responsible for coding the inhibitors, cloning the gene in suitable vectors and cell types, and expressing the gene to produce the inhibitors. Mature recombinant 30 kDa TNF inhibitor and mature recombinant 40 kDa TNF inhibitor have been demonstrated to be capable of inhibiting TNF (EP 393 438, EP 422 339, PCT Publication No. WO 92/16221 and PCT Publication No. WO 95/34326).

A recently identified member of the TNFR family, termed Osteoprotegerin (OPG), is a secreted polypeptide which inhibits osteoclast maturation and markedly increases bone density in transgenic mice expressing the OPG polypeptide. OPG inhibited in vitro the formation of mature osteoclasts from hematopoietic progenitor cells and reduced the extent of bone loss in ovariectomized rats (see co-owned and co-pending U.S. Ser. Nos. 08/577,788, filed Dec. 22, 1995; Ser. No. 08/706,945, filed Sep. 3, 1996; and Ser. No. 08/771,777 filed Dec. 20, 1996). OPG may have benefit in the treatment of osteopenia. PCT Application No. WO96/26217 discloses a polypeptide termed Osteoclastogenesis Inhibitory Factor (OCIF) which is identical to OPG.

OPG comprises two domains having different structural and functional properties. The amino-terminal domain spanning residues 22-194 in the mature polypeptide shows homology to other members of the TNFR family, especially TNFR-2, through conservation of cysteine rich domains characteristic of TNFR family members. The carboxy terminal domain spanning residues 194-401 has no significant homology to any known sequences. Unlike a number of other TNFR family members, OPG appears to be exclusively a secreted protein and does not appear to be synthesized as a membrane associated form. Analysis of OPG by reducing and non-reducing gel electrophoresis indicated that the full-length mature polypeptide of 380 amino acids formed a dimer having a molecular weight of about 120 kDa as compared to the monomer molecular weight of about 60 kDa. OPG polypeptides having certain truncations in the carboxy terminal domain or substitutions of certain cysteine residues within in the carboxy terminal domain formed dimeric OPG to a lesser extent and had lower biological activity compared to wild-type OPG. However, replacement of part or all of the OPG carboxy terminal domain with an Fc region of IgG restored biological activity in the OPG fusion protein to near normal levels. Based upon these observations, the amino-terminal region of OPG appeared to be required for biological activity while the carboxy-terminal domain was important for dimerization. In addition, the biological activity of OPG appeared to be enhanced when the molecule was in dimeric form.

In a therapeutic regimen, it is often desirable to modulate a biological response either by enhancing or blocking a signal received by a receptor. Enhancement of a biological response can involve increasing the affinity of the signalling molecule for a receptor, or increasing the half-life of the molecule in circulation such that it is bound to the receptor for a longer period of time. When the signalling molecule is a polypeptide, enhancement of a biological response may be achieved by constructing analogs which have amino acid sequence changes that increase binding or half-life, derivatives (e.g., polypeptides modified with water soluble polymers) to increase solubility and/or half-life, or chimeric polypeptides (e.g, polypeptides fused to the Fc region of IgG) which increase half-life, solublility and/or modify the aggregation state of the protein in circulation. Similar approaches may be taken to develop therapeutic proteins which act as antagonists by blocking a biological response. In particular, soluble forms of transmembrane receptors which may encompass part or all of the extracellular domains have been used to prevent ligand binding and receptor activation. Soluble receptors have been developed as chemically-modified derivatives and as chimeric polypeptides.

Due to the relatively low inhibition of cytotoxicity exhibited by the 3OkDa,TNF inhibitor and 40 kDa TNF inhibitor (Butler et al. Cytokine 6, 616-623 (1994)), various groups have generated dimers of TNF inhibitor proteins (Butler et al. (1994), suira; and Martin et al. Exp. Neurol. 131, 221-228 (1995)). However, the dimers may generate an antibody response (Martin et al. (1995), supra; and Fisher et al. New Eng. J. Med., 334, 1697-1702 (1996)).

Generation of chimeric polypeptides has been described in the art. For example, construction of hydrid immunoglobulin molecules by fusion of a ligand binding partner to a human IgG chain is described in U.S. Pat. Nos. 5,116,964 and 5,428,130. Construction of a chimeric polypeptide comprising the extracellular domain of a TNF receptor fused to a mouse IgG heavy chain is described in U.S. Pat. No. 5,447,851. Chimeric polypeptides comprising the extracellular domain of a human PDGF receptor fused to dimerizing proteins is described in EP 0 721 983. Multimers of soluble forms of TNF receptors are described in U.S. Pat. No. 5,478,925.

While fusion proteins, such as those comprising immunoglobulin constant regions, may have desirable biological properties, they can elicit an. immune response which limits their usefulness as a human therapeutic.

Therefore, it is an object of the invention to provide chimeric polypeptides which enhance or block a biological response. Such polypeptides may have increased stability, solubility, circulating half-life and decreased immunogenicity.

It is another object of the invention to provide chimeric polypeptides which combine the active region of a signalling molecule with an OPG dimerization domain wherein said chimeric polypeptides will enhance or block a biological response characteristic of the signalling molecule portion of the chimera.

It is another object of the invention to provide OPG chimeric polypeptides which form dimers, trimers and higher multimers which may have advantageous properties such as increased binding affinity, greater stability, and longer circulating half-life compared to monomeric forms.

### SUMMARY OF THE INVENTION

The invention provides for chimeric polypeptides comprising fusions of an OPG dimerization domain to a heterologous sequence. Also provided for are nucleic acid sequences encoding the polypeptides, expression vectors and host cells for production of the polypeptides, and pharmaceutical compositions comprising the polypeptides.

A heterologous sequence of the invention comprises an amino acid sequence of a cell signalling molecule, such as a receptor, an extracellular domain thereof, and an active fragment, derivative and analog of a receptor or an extraceullular domain. In a preferred embodiment, heterologous sequences are selected from the family of TNF-like receptors. Such sequences preferentially include functional extracellular ligand binding domains and lack functional transmembrane and cytoplasmic domains. In another embodiment, the transmembrane and cytoplasmic domains are deleted in whole or in part. It is understood that heterologous sequences of the invention do not include the amino terminal region of OPG defined by residues 22-194 as shown in U.S. Ser. No. 08/577,788 filed Dec. 22, 1995 and hereby incorporated by reference, and do not include related amino acid sequences which, when fused to an OPG dimerization domain, exhibit the biological activity of OPG.

Also encompassed by the invention are multimeric polypeptides comprising covalently associated monomers of OPG chimeric polypeptides. The monomers may have identical heterolgous sequences or different heterologous sequences. In a preferred embodiment, the multimeric polypeptide is a dimer, either a heterodimer (different heterologous sequences) or a homodimer (identical heterologous sequences).

The chimeric polypeptides of the invention are produced by transforming or transfecting host cells with nucleic acids encoding the polypeptide, culturing the host cells, and recovering the polypeptide from the culture. Also provided for are expression vectors and host cells for producing the chimeric polypeptides.

The chimeras are useful for detecting molecules which interact with fused heterologous sequences and thereby identifying potential new receptors and ligands. The compositions of chimeric polypeptides provided herein are useful for treatment of a variety of disorders, for example those related to receptor binding. In one embodiment, compositions comprising TNF/OPG and TNFR/OPG chimeric are used to treat TNF and TNFR mediated disorders, such as inflammation, autoimmune diseases, and disorders related to excessive apoptosis

### DESCRIPTION OF THE FIGURES

FIG. 1. Amino acid sequences of human, mouse and rat OPG dimerization domains (residues 194-401 of corresponding full-length OPG polypeptides). Conserved cysteine residues implicated in disulfide bond formation are underlined.

FIG. 2. Nucleic acid and amino acid sequence of mature, full-length 30 kDa TNF inhibitor.

FIG. 3. Nucleic acid and amino acid sequence of mature, full-length 40 kDa TNF inhibitor.

FIG. 4 Amino acid sequences of TNFbp/OPG chimeric polypeptides. The TNFbp portion of the chimera is the full-length 30 kDa TNF inhibitor with the leader sequence (underlined) and the additional sequence VKGTEDSGTT at the carboxy terminus. OPG dimerization domains are human OPG residues 194-401, 196-401, 217-401, 248-401 and 304-401. The junction of the TNFbp and OPG sequences creates an Age I restriction site in the DNA sequence and adds a glycine codon (at position 212).

FIG. 5. Gel electrophoresis analysis of TNFbp/OPG chimeric polypeptides. TNFbp/OPG chimeic plasmids were transfected into CHO d-cells. supernatants from serum-free roller bottle harvests were analyzed on a 12% polyacrylamide, Tris-glycine, non-reducing gel. Dimerization patterns were compared to a TNFbp-Fc fusion (lane 1) and TNFbp monomer (lane 8).

FIG. 6. Inhibition of TNFα cytotoxicity on L929 cells. Serum-free conditioned medium samples of TNFbp/Fc and TNFbp/OPG[194-401] fusion polypeptides were serially diluted and assayed for inhibition of TNFα cytotoxicity on L929 cells.

### DETAILED DESCRIPTION OF THE INVENTION

The invention provides for a chimeric polypeptide comprising a fusion of an OPG dimerization domain to a heterologous sequence.

The term “heterologous sequence” refers to an amino acid sequence which is involved in cell signalling and acts to modulate cell growth, differentiation or metabolism. In general, heterologous sequences comprise extracellular ligand binding domains of cell surface receptors and their cognate ligands. When present as part of an OPG chimeric polypeptide, a heterologous sequence of the invention comprises about ten or more amino acids in length, about 20 or more amino acids in length, about 50 or more amino acids in length, and about 100 or more amino acids in length. A heterologous sequence will be of sufficient size to confer on a chimeric polypeptide a functional property such as receptor binding, enzymatic activity, inhibitor activity and the like; however, it is understood that the chimeric polypeptides will not have functional properties identical to OPG although they may share one or more functions in common with OPG. Heterologous sequences may encode full-length polypeptides or active fragments, derivatives and analogs thereof.

In preferred embodiments, chimeric OPG polypeptides include heterologous sequences encoding growth factors, cytokines, hormones, cell adhesion molecules and other polypeptide factors which are typically secreted. Chimeric OPG polypeptides also include heterologous sequences which encode receptors for growth factors, cytokines, hormones, cell adhesion molecules, and the like, and preferably will include extracellular ligand binding domains from said receptors, and active fragments, derivatives and analogs thereof. The heterologous sequences may or may not be capable of forming dimers or higher aggregates when the sequences are present in a naturally occurring form.

The “OPG dimerization domain” refers to that portion of the OPG polypeptide which is capable of forming covalently associated multimeric polypeptides. It is understood, however, that chimeric polypeptides comprising an OPG dimerization domain are not restricted to forming dimers, but may form higher multimers as well (trimers, tetramers, etc.) The domain may have the amino acid sequence of the human osteoprotegerein dimerization domain, or it may be a fragment, derivative or analog thereof which is capable of forming covalently associated multimers. More specifically, an OPG dimerization domain will retain one or more cysteine residues which will allow formation of at least one interchain disulfide bond. In a preferred embodiment, the OPG dimerization domain has the amino acid sequence from about residues 194 to 401 inclusive of human OPG.

As used herein, the term “fragment” comprises a deletion of one or more amino acids in a heterologous sequence or in an OPG dimerization domain. The deletion may occur at the amino terminal end, the carboxy terminal end or in an internal region of the sequence. As used herein, the term “derivative” refers to a modification of the polypeptide backbone of an OPG chimera, either within the OPG dimerization domain or within the heterologous sequence. Said modificaitons include, but are not limited to, attachment of water soluble polymers, hydrophobic moieties, fluorescent tags, enzymatic labels and the like. As used herein, the term “analogs” refers to one or more amino acid substitutions and/or insertions within a polypeptide. Substitutions may involve conservative replacements or non-conservative replacements of amino acids which are known to one skilled in the art. Amino acid insertions may occur at the amino or carboxy terminal ends of either the OPG dimerization domain or the heterolgous sequence or both, or may occur in internal regions.

Polypeptides

Chimeric polypeptides of the invention comprise a heterologous sequence fused at its carboxy terminus to the amino terminus an OPG dimerization domain or, alternatively, an OPG dimerization domain fused at its carboxy terminus to the amino terminus of a heterologous sequence. Chimeric polypeptides may be constructed as a direct fusion of a heterologous sequence and an OPG dimerization domain or may be constructed with a spacer or adapter region having one or more amino acids inserted between the two portions of the polypeptide. Optionally, the spacer region may encode a protease cleavage site. The precise site of the fusion is not critical and may be varied by one skilled in the art in order to optimize binding charcteristics and/or biological activity of the heterologous sequence.

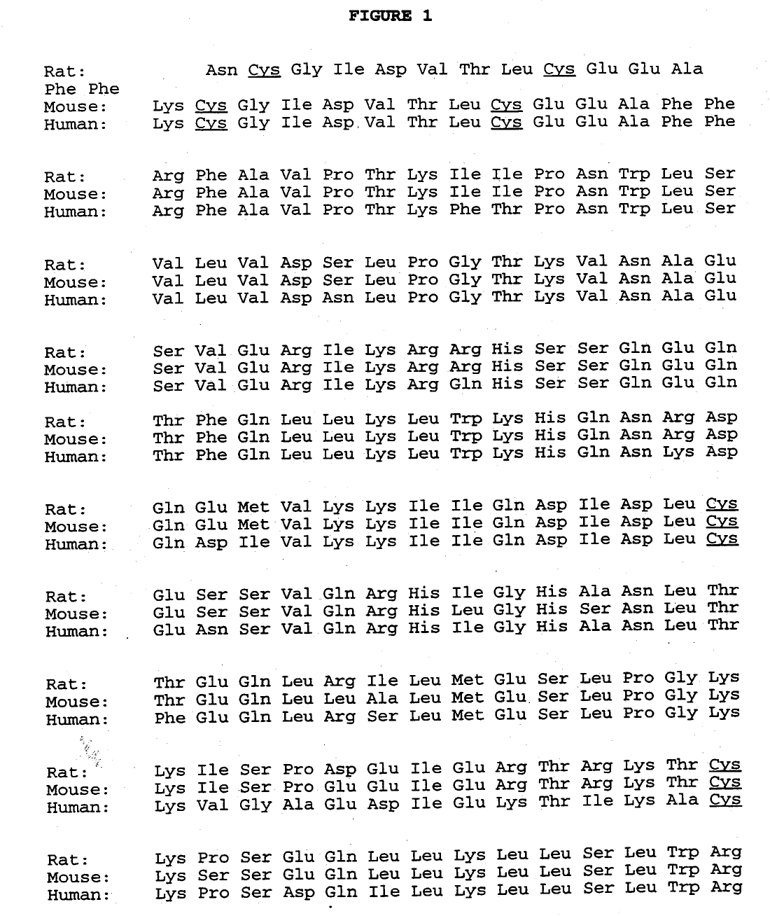

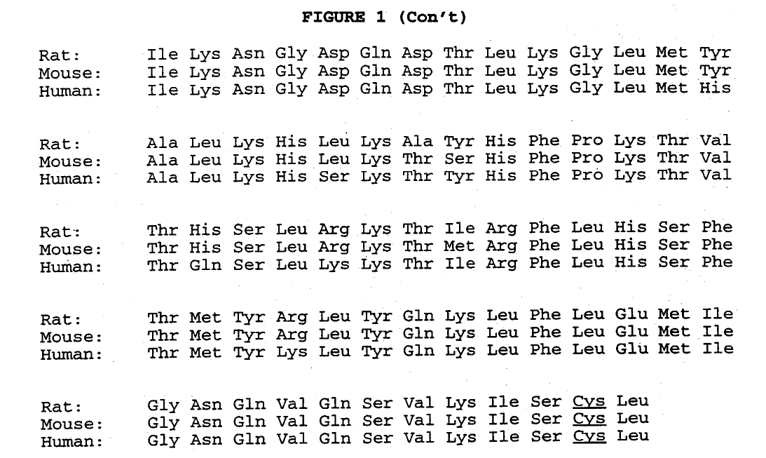

According to the invention, an OPG dimerization domain may be mammalian in origin (such as from mouse, rat or human) or may be a fragment or analog thereof which is capable of forming covalently associated dimers or higher order multimers. The amino acid sequences of rat, mouse and human OPG dimerization domains span from about residues 194-401 of their respective full-length OPG oolypeptides as shown in FIG. 1 (SEQ ID NO:______). Fragments and analogs of an OPG dimerization domain include: deletion or substitution of a cysteine residue at any of positions 195, 202, 277, 319 and 400; addition of one or more cysteine residues; rearrangement of the configuration of cysteine residues which may entail a net increase from, a net decrease from, or no change in the number of cysteine residues compared to residues 194-401 of the human OPG dimerization domain; amino-terminal truncations of OPG[194-401], e.g, 195-401, 196-401, and so forth; C-terminal truncations of OPG[194-401], e.g, 194-400, 194-399, and so forth; conservative substitutions of amino acid residues in OPG[194-401] wherein the substitutions comprise replacements with structurally or functionally similar amino acids which are known to one skilled in the art; and any combinations thereof.

Heterologous sequences which form part of a chimeric OPG polypeptide include receptors having known extracellular ligand binding domains. Examples are receptor protein-tyrosine kinases, such as the platelet-derived growth factor receptor (PDGFR) family, fibroblast growth factor receptor (FGFR) family, insulin receptor family, epidermal growth factor receptor (EGFR) family, nerve growth factor (NGFR) family, hepatocyte growth factor family (HGFR), EPH family, AXL family, TIE family, DDR family, ROR family, and other receptor protein tyrosine kinases (see van der Geer et al. Ann. Rev. Cell Biol. 10, 251-337 (1994)). Other examples of receptors having extracellular ligand binding domains include the cytokine receptor superfamily, such as G-CSF, GM-CSF (α and β subunits), MGF; EPO, MGDF, IL-1, IL-2, IL-3, IL-4, IL-5, IL-6, IL-7, IL-9, IL-l1, growth hormone, α-interferon, β-interferon, and γ-interferon receptors, the seven transmembrane domain receptor superfamily, such as acetylcholine, adrenergic, dopamine, thrombin, FSH, gonadotropin, thyrotropoin, clacitonin and parathyroid hormone receptors, and cell adhesion-receptors. It is understood that the receptors cited herein are merely examples and that heterologous sequences present in OPG chimeric polypeptides are not limited to the above-mentioned receptors.

Other heterologous sequences of the invention comprise growth factors, hormones, cytokines, cell adhesion proteins and the like. Also included are corresponding ligands for the receptor protein tyrosine kinases, ligands for cytokine receptors, ligands for seven transmembrane domain receptors, and ligands for cell adhesion receptors.

In a preferred embodiment, the heterologous sequence is a member of the TNF receptor superfamily or is derived from a member of the TNF receptor family. Members include TNFR-1, TNFR-2, TNFrp, NGFR, FasB, CD40, OX40, CD27, CD30, and 4-1BB. Typically the extracellular domains of TNF receptors, or active fragments, derivatives and analogs thereof, are fused to an OPG dimerization domain. Active fragments of TNF receptors will have at least one cysteine rich domain, alternatively two, three or four cysteine rich domains, or alternatively one, two or three cysteine rich domains and a portion thereof, for example, two cysteine rich domains and a portion of a third domain. Activity of a TNF/OPG chimeric polypeptide may include biological activity or ligand binding activity characteristic of a TNF family member which may be evaluated using procedures known to one skilled in the art.

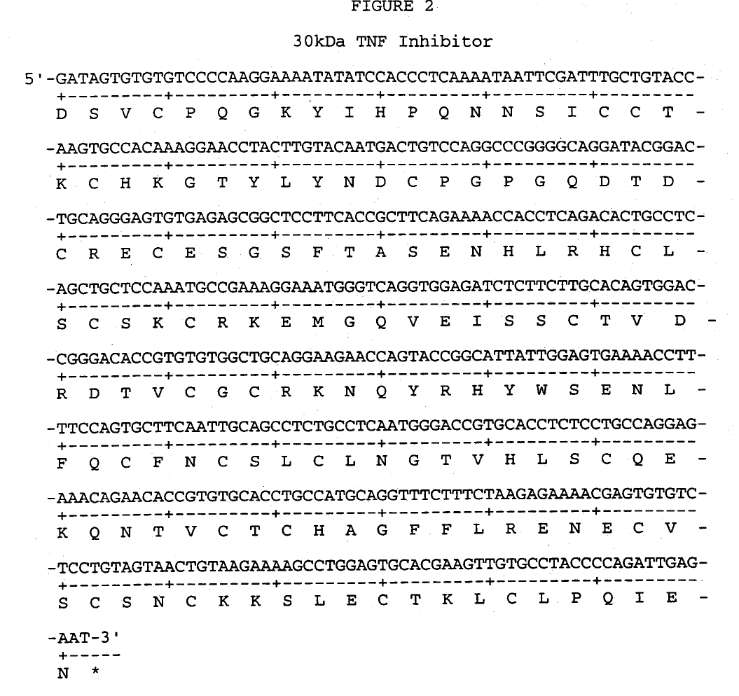

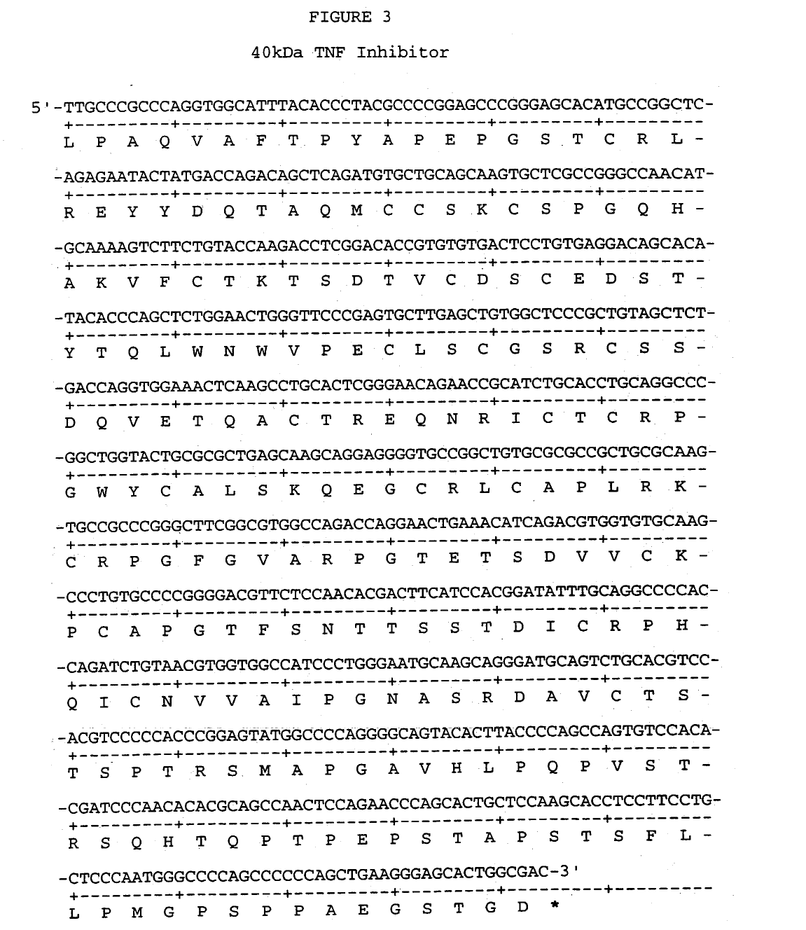

Preferred heterologous sequences comprise TNFR-1 or are derived from TNFR-1, and may be a 30 kDa TNF inhibitor, a 40 kDa TINF inhibitor, or a functionally active low molecular weight TNF inhibitor. The nucleic acid and amino acid sequence of mature, full-length 30 kDa TNF inhibitor is shown in FIG. 2 (SEQ ID NO:______). The nucleic acid and amino acid sequence of mature, full-length 40 kDa TNF inhibitor is shown in FIG. 3 (SEQ ID NO:______). The low molecular weight TNF inhibitors are modified forms of the 30 kDa TNF inhibitor and 40 kDa TNF inhibitor which do not contain the fourth domain (amino acid residues Thr¹²⁷—Thr¹⁶¹ of the 30 kDa TNF inhibitor and amino acid residues Pro¹⁴¹—Thr¹⁷⁹ of the 40 kDa TNF inhibitor); a portion of the third domain (amino acid residues Asn¹¹¹—Cys¹²⁶ of the 30 kDa TNF inhibitor and amino acid residues Pro¹²³—Lys140 of the 40 kDa TNF inhibitor); and, optionally, which do not contain a portion of the first domain (amino acid residues Asp¹—Lys²¹ of the 30 kDa TNF inhibitor and amino acid residues Leu¹—Lys³⁴ of the 40 kDa TNF inhibitor).

The heterologous sequences of the present invention include derivatives of TNFR-1 proteins represented by the formula R₁-[Cys¹⁹—Cys¹⁰³]-R₂ and R₄-[Cys³²—Cys¹¹²]-R₅. These proteins are deletion variants of the 30 kDa TNF inhibitor and the 40 kDa TNF inhibitor, respectively, and are referred to as “truncated TNFbp(s)”.

By “R₁-[Cys¹⁹—Cys¹⁰³]-R₂” is meant one or more proteins wherein [Cys¹⁹—Cys¹⁰³] represents residues 19 through 103 of mature, full-length 30 kDa TNF inhibitor, the amino acid residue numbering scheme of which is provided in FIG. 2 (SEQ ID NO:______) to facilitate the comparison; wherein R₁ represents a methionylated or nonmethionylated amine group of Cys¹⁹ or of amino-terminus amino acid residue(s) selected from the group:

| | |
|---|---|
| C | |
| IC | |
| SIC | |
| NSIC | (SEQ ID NO:_) |
| NNSIC | (SEQ ID NO:_) |
| QNNSIC | (SEQ ID NO:_) |
| PQNNSIC | (SEQ ID NO:_) |
| HPQNNSIC | (SEQ ID NO:_) |
| IHPQNNSIC | (SEQ ID NO:_) |
| YIHPQNNSIC | (SEQ ID NO:_) |
| KYIHPQNNSIC | (SEQ ID NO:_) |
| GKYIHPQNNSIC | (SEQ ID NO:_) |
| QGKYIHPQNNSIC | (SEQ ID NO:_) |
| PQGKYIHPQNNSIC | (SEQ ID NO:_) |
| CPQGKYIHPQNNSIC | (SEQ ID NO:_) |
| VCPQGKYIHPQNNSIC | (SEQ ID NO:_) |
| SVCPQGKYIHPQNNSIC | (SEQ ID NO:_) |
| DSVCPQGKYIHPQNNSIC; | (SEQ ID NO._) |

and wherein R₂ represents a carboxy group of Cys¹⁰³ or of carboxy-terminal amino acid residues selected from the group:

| | |
|---|---|
| F | |
| FC | |
| FCC | |
| FCCS | (SEQ ID NO:_) |
| FCCSL | (SEQ ID NO:_) |
| FCCSLC | (SEQ ID NO:_) |
| FCCSLCL; | (SEQ ID NO:_) |

and variants thereof.

Exemplary tumor necrosis factor binding proteins which comprise TNFbp/OPG chimeric polypeptides of the present invention include the following molecules: NH₂-MDSVCPQGKYIHPQNNSIC-[Cys¹⁹—Cys¹⁰³]-FC—COOH (also rererred to as 30 kDa TNFbp 2.6C105); NH₂-MDSVCPQGKYIHPQNNSIC-[Cys¹⁹—Cys¹⁰³]-FNCSL—COOH (also referred to as 30 kDa TNFbp 2.6C106); NH₂-MDSVCPQGKYIHPQNNSIC-[Cys¹⁹—Cys103]-FNCSL—COOH (also referred to as 30 kDa TNFbp 2.6N105); NH₂-MYIHPQNNSIC-[Cys¹⁹—Cys¹⁰³]-FNCSL—COOH (also referred to as 30 kDa TNFbp 2.3d8); NH₂—M-[Cys¹⁹-Cys¹⁰³]-FNCSL—COOH (also referred to as 30 kDa TNFbp 2.3d18) and NH₂-MSIS-[Cys¹⁹—Cys¹⁰³]-FNCSL—COOH (also referred to as 30 kDa TNFbp 2.3d15), either methionylated or nonmethionylated, and variants and derivatives thereof.

By “R₄-[Cys³²—Cys112]-R₅” is meant one or more proteins wherein [Cys³²—Cys¹¹²] represents residues Cys³² through Cys¹¹² of mature, full-length 40 kDa TNF inhibitor, the amino acid residue numbering scheme of which is provided in FIG. 3 (SEQ ID NO:______) to facilitate the comparison; wherein R₄ represents a methionylated or nonmethionylated amine group of Cys³² or of amino-terminus amino acid residue(s) selected from the group:

| | |
|---|---|
| C | |
| MC | |
| QMC | |
| AQMC | (SEQ ID NO:_) |
| TAQMC | (SEQ ID NO:_) |
| QTAQMC | (SEQ ID NO:_) |
| DQTAQMC | (SEQ ID NO:_) |
| YDQTAQMC | (SEQ ID NO:_) |
| YYDQTAQMC | (SEQ ID NO:_) |
| EYYDQTAQMC | (SEQ ID NO:_) |
| REYYDQTAQNC | (SEQ ID NO:_) |
| LREYYDQTAQMC | (SEQ ID NO:_) |
| RLREYYDQTAQMC | (SEQ ID NO:_) |
| CRLREYYDQTAQMC | (SEQ ID NO:_) |
| TCRLREYYDQTAQMC | (SEQ ID NO:_) |
| STCRLREYYDQTAQMC | (SEQ ID NO:_) |
| GSTCRLREYYDQTAQMC | (SEQ ID NO:_) |
| PGSTCRLREYYDQTAQMC | (SEQ ID NO:_) |
| EPGSTCRLREYYDQTAQMC | (SEQ ID NO:_) |
| PEPGSTCRLREYYDQTAQMC | (SEQ ID NO:_) |
| APEPGSTCRLREYYDQTAQMC | (SEQ ID NO:_) |
| YAPEPGSTCRLREYYDQTAQMC | (SEQ ID NO:_) |
| PYAPEPGSTCRLREYYDQTAQMC | (SEQ ID NO:_) |
| TPYAPEPGSTCRLREYYDQTAQMC | (SEQ ID NO:_) |
| FTPYAPEPGSTCRLREYYDQTAQMC | (SEQ ID NO:_) |
| AFTPYAPEPGSTCRLREYYDQTAQMC | (SEQ ID NO:_) |
| VAFTPYAPEPGSTCRLREYYDQTAQMC | (SEQ ID NO:_) |
| QVAFTPYAPEPGSTCRLREYYDQTAQMC | (SEQ ID NO:_) |
| AQVAFTPYAPEPGSTCRLREYYDQTAQMC | (SEQ ID NO:_) |
| PAQVAFTPYAPEPGSTCRLREYYDQTAQMC | (SEQ ID NO:_) |
| LPAQVAFTPYAPEPGSTCRLREYYDQTAQMC; | (SEQ ID NO:_) |

and wherein R₅ represents a carboxy group of Cys¹¹² or of carboxy-terminal amino acid residues selected from the group:

| | |
|---|---|
| R | |
| RL | |
| RLC | |
| RLCA | (SEQ ID NO:_) |
| RLCAP | (SEQ ID NO:_) |
| RLCAPL | (SEQ ID NO:_) |
| RLCAPLR | (SEQ ID NO:_) |
| RLCAPLRK | (SEQ ID NO:_) |
| RLCAPLRKC | (SEQ ID NO:_) |
| RLCAPLRKCR | (SEQ ID NO:_) |

and variants thereof.

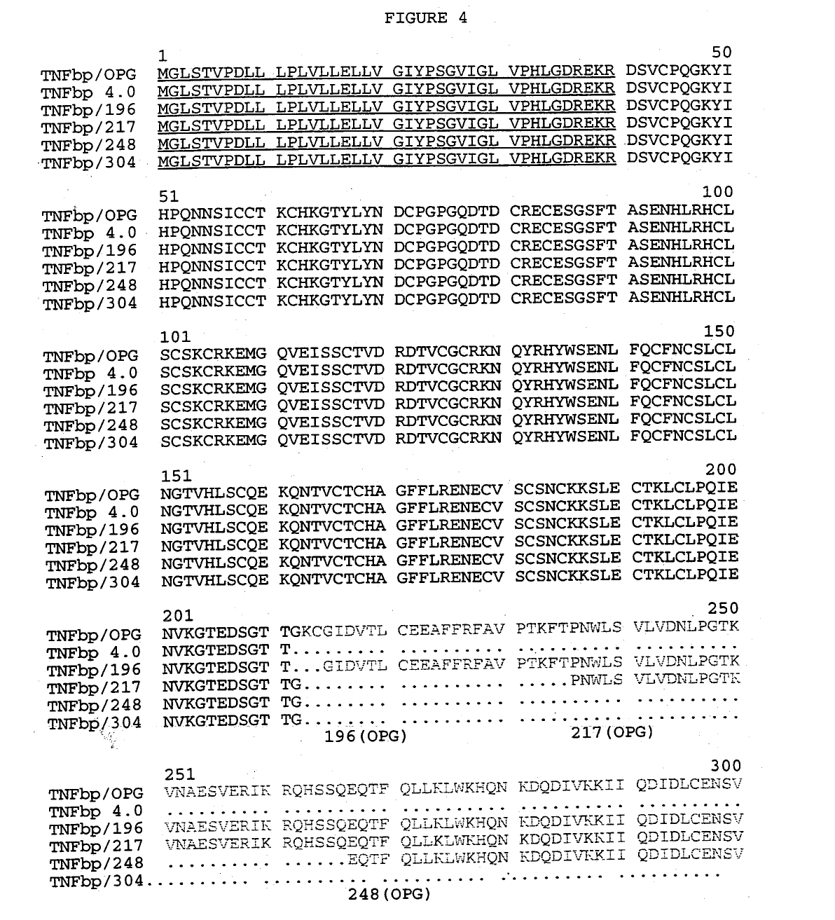

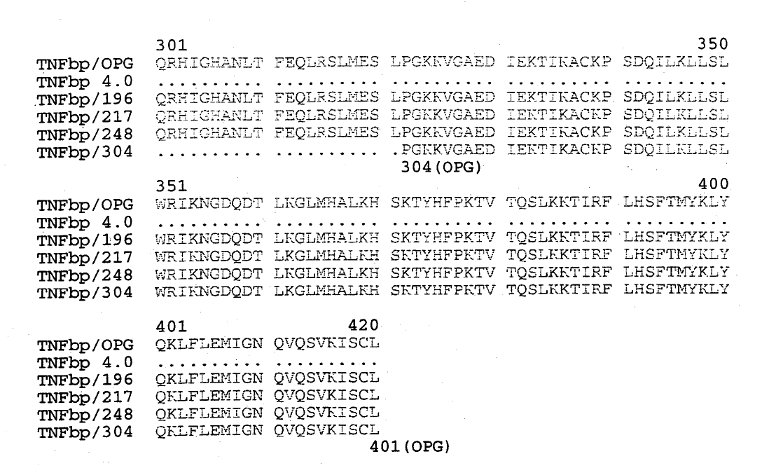

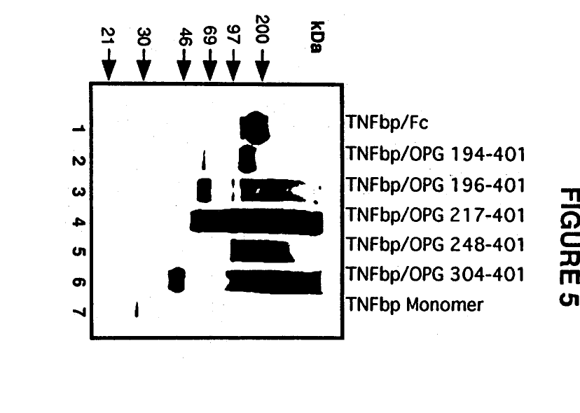

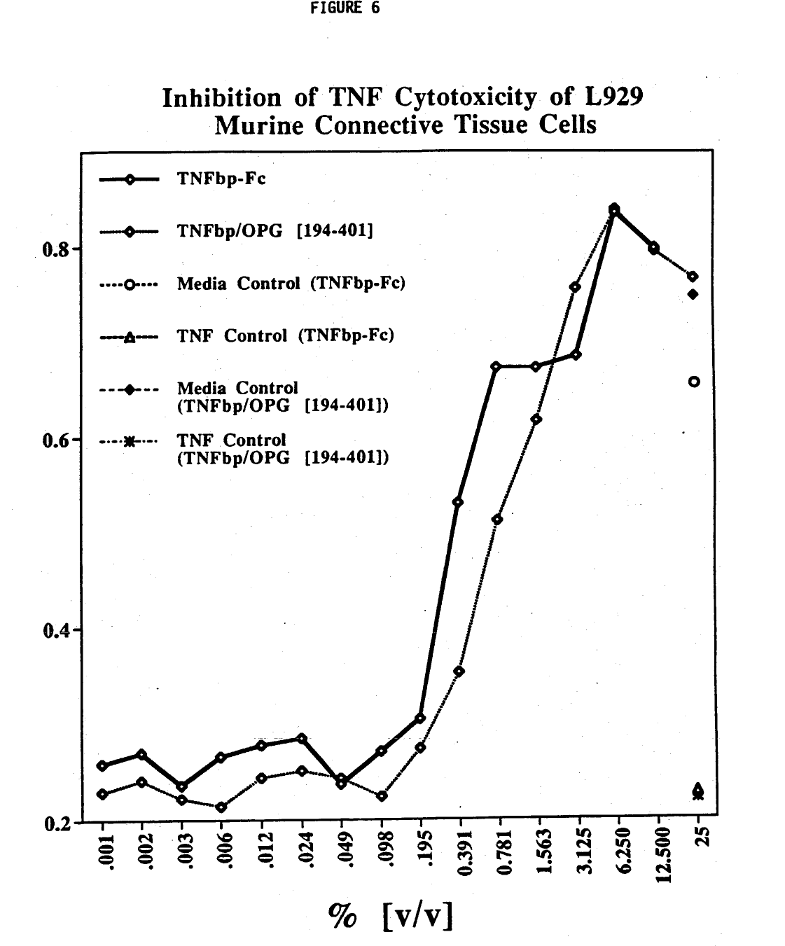

As shown in Example 1, a hybrid DNA molecule encoding TNFbp 4.0, the full-length 30 kDa TNF inhibitor (FIG. 2) with the additional sequence VKGTEDSGTT extending from the carboxy terminus, and human OPG[194-401] was constructed. The resulting chimeric polypeptide, termed TNFbp/OPG[194-401] has the amino acid sequence as shown in FIG. 4. Upon expression, the mature chimeric polypeptides formed dimers in conditioned medium of transfected host cells as determined by non-reducing SDS-PAGE (see FIG. 5). Additional TNFbp fusions were constructed to amino terminal truncations of the human OPG dimerization domain. These constructs are designated TNFbp/OPG[196-401], TNFbp/OPG[217-401], TNFbp/OPG[248-401], and TNFbp/OPG[304-401] and the amino acid sequences are shown in FIG. 4. OPG[194-401] has the full complement of five cysteine residues which are involved in covalent association of OPG dimerization domains. OPG[196-401] lacks one cysteine residue at position 195, OPG[217-401] and OPG[248-401] lacks a second cysteine residue at position 202, and OPG[304-401] lacks a third cysteine residue at position 277 (see FIG. 1 for location of cysteine residues). The chimeric polypeptides produced in conditioned medium of transfected CHOd-host cells were analyzed by non-reducing SDS-PAGE (FIG. 5). In the L929 cytotoxicity assay, the TNFbp/OPG[194-401] chimera showed activity similar to a TNFbp/Fc chimera (FIG. 6).

The invention also provides for chimeric OPG polypeptides which form multimers (i.e., dimers, trimers and higher multimers). Multimers of the invention comprise covalently associated monomeric OPG chimeras wherein the monomers may have identical heterologous sequence or different heterologous sequences. Preferably, the chimeric polypeptides are dimers or trimers. Preparations of multimeric polypeptides will be essentially free of monomeric OPG chimeras which are not covalently associated and of inactive multimers. Such preparations are made using techniques available to one skilled in the art

Modifications of chimeric OPG polypeptides are encompassed by the invention and include post-translational modifications (e.g., N-linked or O-linked carbohydrate chains, processing of N-terminal or C-terminal ends), attachment of chemical moieties to the amino acid backbone, chemical modifications of N-linked or O-linked carbohydrate chains, and addition of an N-terminal methionine residue as a result of procaryotic host cell expression. The polypeptides may also be modified with a detectable label, such as an enzymatic, fluorescent, isotopic or affinity label to allow for detection and isolation of the protein.

Also provided by the invention are chemically modified derivatives of OPG which may provide additional advantages such as increased solubility, stability and circulating time of the polypeptide, or decreased immunogenicity (see U.S. Pat. No. 4,179,337). The chemical moieties for derivitization may be selected from water soluble polymers such as polyethylene glycol, ethylene glycol/propylene glycol copolymers, carboxymethylcellulose, dextran, polyvinyl alcohol and the like. The polypeptides may be modified at random positions within the molecule, or at predetermined positions within the molecule and may include one, two, three or more attached chemical moieties.

The polymer may be of any molecular weight, and may be branched or unbranched. For polyethylene glycol, the preferred molecular weight is between about lkDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog).

The polyethylene glycol molecules (or other chemical moieties) should be attached to the protein with consideration of effects on functional or antigenic domains of the protein. There are a number of attachment methods available to those skilled in the art, e.g. EP 0 401 384 herein incorporated by reference (coupling PEG to G-CSF), see also Malik et al. Exp. Hematol. 20, 1028-1035 (1992) (reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having, a free carboxyl group may include aspartic acid residues glutamic acid residues and the C-terminal amino acid residue. Sulfhydrl groups may also be used as a reactive group for attaching the polyethylene glycol molecule(s). Preferred for therapeutic purposes is attachment at an amino group, such as attachment at the N-terminus or lysine group.

One may specifically desire N-terminally chemically modified protein. Using polyethylene glycol as an illustration.of the present compositions, one may select from a variety of polyethylene glycol molecules (by molecular weight, branching, etc.), the proportion. of polyethylene glycol molecules to protein (or peptide) molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective N-terminal chemically modification may be accomplished by reductive alkylation which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved.

The chimeric OPG polypeptides of the invention are isolated and purified from other constituents present in lysates or supernatants of host cells expressingthe polypeptides. In one embodiment, the polypeptide is free from association with other human proteins, such as the expression product of a bacterial host cell. Also provided by the invention is a method for the purification of OPG chimeric polypeptides. The purification process may employ one or more standard protein purification steps in an appropriate order to obtain purified protein. The chromatography steps can include ion exchange, gel filtration, hydrophobic interaction, reverse phase, chromatofocusing, affinity chromatography employing an anti-OPG antibody or biotin-streptavidin affinity complex and the like. When preparations of selected multimeric OPG chimeras are desired, the purification method may be carried out to separate species of different aggregation states, for example, separation of monomeric from dimeric OPG chimeras, or separation of dimeric from tetrameric OPG chimeras.

Chimeric OPG polypeptides may be used in assays to screen for binding molecules. Examples of such molecules include, but are not limited to, nucleic acids, polypeptides, small molecular weight peptides, carbohydrates, lipids and small molecular weight organic compounds. Assays will employ combining candidate molecules (either purified or unpurified) with chimeric OPG polypeptides under conditions that allowing binding, and measuring the extent of binding to the chimeric polypeptide. Binding measurements are made using detection systems available to one skilled in the art, such as radioactivity, enzymatic activity, fluorescence, and surface plasmon resonance.

Nucleic Acids

The invention provides for an isolated nucleic acid encoding a chimeric polypeptide having an OPG dimerization domain fused to a heterologous sequence. The nucleic acids encode a chimeric OPG polypeptide wherein the heterologous sequence is a cell signalling molecule such as a receptor or a receptor, ligand. In a preferred embodiment, the heterologous nucleic acid sequence encodes a polypeptide of the TNFR family, or a fragment, derivative or analog thereof, provided however that the heterologous nucleic acid sequence does not encode OPG[22-194] as shown in U.S. Ser. No. 08/577,788 filed Dec. 22, 1995, or a homologous sequence which, when fused to an OPG dimerization domain, has the biological activity of OPG.

The nucleic acids of the invention encode chimeric OPG polypeptides selected from the following:

a) the nucleic acid sequences which encode the polypeptides shown in FIG. 1 (SEQ ID NO:______) or complementary strands thereof; and

b) the nucleic acids sequences which hybridize under high stringency conditions with the sequences in (a), and degenerate sequences thereof, provided however that the polypeptides do not have the biological activity of OPG. Nucleic acids encoding OPG chimeric polypeptides may hybridize over part or all of the nucleic acid sequences encoding the OPG dimerization domains shown in FIG. 1 (SEQ ID NO:______).

The conditions for hybridization are generally of high stringency using temperatures, solvents and salt concentrations wherein the hydridizing sequences are about 12-20° C. below the melting temperature (Tₘ) of the perfectly matched duplex. Equivalent stringency to these conditions may be readily ascertained by one skilled in the art by adjusting salt and organic solvent concentrations and temperature. Specific hybridization conditions are described in Sambrook et al. Molecular Cloning: A Laboratory Manual, 2nd ed. Cold Spring Harbor Laboratory Press, Cold Spring Harbor, N.Y. (1989)

Preferred sequences include nucleic acids which encode chimeric OPG polypeptides having rat, mouse and human OPG dimerization domains. DNA encoding human OPG dimerization domain was provided in a full-length human OPG plasmid designated pRcCMV—human OPG and deposited with the American Type Culture Collection, Rockville, Md. on Dec. 27, 1995 under accession no. 69969. DNA encoding rat OPG dimerization domain was provided in a full-length rat OPG plasmid designated pMOB-B1.1 and depositpd with the American Type Culture Collection, Rockville, Md. on Dec. 27, 1995 under ATCC accession no. 69970. DNA encoding mouse OPG dimerization domain was provided in a full-length mouse OPG plasmid designated pRcCMV-murine OPG and deposited with the American Type Culture Collection, Rockville, Md. on Dec. 27, 1995 under accession no. 69971. The nucleic acids of the invention will hybridize under-stringent conditions to the DNA inserts of ATCC accession nos. 69969, 69970, and 69971.

In a preferred embodiment, heterologous sequences will comprise nucleic acids encoding TNFR-1, and fragments, derivatives and analogs thereof, such as the TNF 30 kDa inhibitor or TNF 40 kDa inhibitor. Presently preferred heterologous sequences include those nucleic acids encoding 30 kDa TNFbp 2.6C105, 30 kDa TNFbp 2.6C106, 30 kDa TNFbp 2.6N105, 30 kDa TNFbp 2.3d8, 30 kDa TNFbp 2.3d18 and 30 kDa TNFbp 2.3d15.

Also provided by the invention are nucleic acids encoding variants of an OPG chimeric polypeptide wherein the variations may be in the heterologous sequence or the OPG dimerization domain or both. The nucleic acid derivatives comprise addition, substitution, insertion or deletion of one or more nucleotides such that the resulting sequences encode chimeric OPG polypeptides comprising one or more amino acid residues which have been added, deleted, inserted or substituted in either the heterologous sequence or the OPG dimerization domain or both. The nucleic acid derivatives may be naturally occurring, such as by splice variation or polymorphism, or may be constructed using site-directed mutagenesis techniques available to the skilled worker. Chimeric OPG polypeptide variants are described in the previous section entitled “Polypeptides” and it is anticipated that nucleic acids encoding all variants disclosed therein, and degenerate molecules thereof, are encompassed by the invention.

Examples of the nucleic acids of the invention include cDNA, genomic DNA, synthetic DNA and RNA. cDNA is obtained from libraries prepared from mRNA isolated from various tissues expressing OPG. In humans, tissue sources for OPG include kidney, liver, placenta and heart. Genomic DNA encoding OPG is obtained from genomic libraries which are commercially available from a variety of species. Synthetic DNA is obtained by chemical synthesis of overlapping. oligonucleotide fragments followed by assembly of the fragments to reconstitute part or all of the coding region and flanking sequences (see U.S. Pat. No. 4,695,623). RNA may be obtained in large quantities use of procaryotic expression vectors which direct high-level synthesis of MRNA, such as vectors using T7 promoters and RNA polymerase.

Nucleic acid sequences of the invention are useful for the expression of chimeric OPG polypeptides. Expression may be carried out in transfected host-cells for production of recombinant protein in quantities sufficient for diagnostic or therapeutic applications. In addition, chimeric OPG polypeptides may be expressed in vivo and secreted into the circulation to provide therapeutic benefit.

Vectors and Host Cells

Expression vectors containing nucleic acid sequences encoding OPG fusion proteins, host cells transformed with said vectors and methods for the production of OPG fusion proteins are also provided by the invention. An overview of expression of recombinant proteins is found in Methods of Enzymology v. 185, Goeddel, D. V. ed. Academic Press (1990).

Host cells for the production of OPG fusion proteins include procaryotic host cells, such as E. coli, yeast, plant, insect and mammalian host cells. E. coli strains such as HB101 or JM101 are suitable for expression. Preferred mammalian host cells include COS, CHOd-, 293, CV-1, 3T3, baby hamster kidney (BHK) cells and others. Mammalian host cells are preferred when post-translational modifications, such as glycosylation and polypeptide processing, are important for OPG chimera activity. Mammalian expression allows for the production of secreted polypeptides which may be recovered from the growth medium.

Vectors for the expression of OPG chimeric polypeptides contain at a minimum sequences required for vector propagation and for expression of the cloned insert. These sequences include a replication origin, selection marker, promoter, ribosome binding site, enhancer sequences, RNA splice sites and transcription termination site. Vectors suitable for expression in the aforementioned host cells are readily available and the nucleic acids of the invention are inserted into the vectors using standard recombinant DNA techniques. Vectors for tissue-specific expression of OPG chimeric polypeptides are also included. Such vectors include promoters which function specifically in liver, kidney or other organs for production in mice, and viral vectors for the expression of OPG in targeted human cells.

Using an appropriate host-vector system, OPG chimeric polypeptides are produced recombinantly by culturing a host cell transformed with an expression vector containing nucleic acid sequences encoding an OPG chimeric polypeptide under conditions such that the polypeptide is produced, and isolating the product of expression. OPG chimeras are produced in the supernatant of transfected mammalian cells or in inclusion bodies of transformed bacterial host cells. OPG chimeras so produced may be purified by procedures known to one skilled in the art as described below. Expression vectors for mammalian hosts are exemplified by plasmids such as pDSRα described in PCT Application No. 90/14363; see also Methods in Enzymology vol. 185, D. V. Goeddel, ed. pp. 487-511 for additional examples. A variety of expression vectors are available for bacterial host cells and are described in Methods in Enzymology, ibid. pp. 14-37 and references cited therein. It is anticipated that the specific plasmids and host cells described are for illustrative purposes and that the choice of any specific plasmid and host cell for expression of an OPG chimeric polypeptide will depend upon consideration of a variety of factors by one skilled in the art.

Antibodies

Also encompassed by the invention are antibodies specifically binding to an OPG chimeric polypeptide. Antigens for the generation of antibodies may be full-length polypeptides or peptides spanning a portion of the OPG sequence. Immunological procedures for the generation of polyclonal or monoclonal antibodies reactive with OPG are known to one skilled in the art (see, for example, Harlow and Lane, Antibodies: A Laboratory Manual Cold Spring Harbor Laboratory Press, Cold Spring Harbor N.Y. (1988)). Antibodies so produced are characterized for binding specificity and epitope recognition using standard enzyme-linked immunosorbent assays. Antibodies also include chimeric antibodies having variable and constant domain regions derived from different species. In one embodiment, the chimeric antibodies are humanized antibodies having murine variable domains and human constant domains. Also encompassed are complementary determining regions grafted to a human framework (so-called CDR-grafted antibodies). Chimeric and CDR-grafted antibodies are made by recombinant methods known to one skilled in the art. Also encompassed are human antibodies made in mice.

Anti-OPG chimera antibodies of the invention may be used as an affinity reagent to purify OPG from biological samples. In one method, the antibody is immobilized on CNBr-activated Sepharose and a column of antibody-Sepharose conjugate is used to remove OPG from liquid samples. Antibodies are also used as diagnostic reagents to detect and quantitate OPG in biological samples by methods described below.

Pharmaceutical Compositions

The invention also provides for pharmaceutical compositions comprising a therapeutically effective amount of an OPG chimeric polypeptide together with a pharmaceutically acceptable diluent, carrier, solubilizer, emulsifier, preservative and/or adjuvant. The term “therapeutically effective amount” refers to an amount which provides a therapeutic effect for a specified condition and route of administration. The composition may be in a liquid or lyophilized form and comprises a diluent (Tris, acetate or phosphate buffers) having various pH values and ionic strengths, solubilizer such as Tween or Polysorbate, carriers such as human serum albumin or gelatin, preservatives such as thimerosal or benzyl alcohol, and antioxidants such as ascrobic acid or sodium metabisulfite. Also encompassed are compositions comprising OPG chimeric polypeptides modified with water soluble polymers to increase solubility or stability. Compositions may also comprise incorporation of OPG chimeric polypeptides into liposomes, microemulsions, micelles. or vesicles for controlled delivery over an extended period of time. Selection of a particular composition will depend upon a number of factors, including the condition being treated, the route of administration and the pharmacokinetic parameters-desired. A more extensive survey of components suitable.for pharmaceutical compositions is found in Remington's Pharmaceutical Sciences, 18th ed. A. R. Gennaro, ed. Mack, Easton, Pa. (1980).

Compositions of the invention may be administered by injection, either subcutaneous, intravenous or intramuscular, or by oral, nasal, pulmonary or rectal administration. The route of administration eventually chosen will depend upon a number of factors and may be ascertained by one skilled in the art.

Pharmaceutical compositions of chimeric OPG polypeptides are useful for treatment of receptor-mediated disorders, for example disorders resulting from the function (or lack thereof) of protein tyrosine kinases, cytokine, seven transmembrane domain, and cell adhesion receptors. Disorders resulting from the function (or lack thereof) of the corresponding polypeptide ligands of the above referenced receptors may-also be treated. In one embodiment, compositions comprising TNF/OPG chimeras are used to treat TNF-related disorders such as inflammation, autoimmune diseases and conditions marked by excessive apoptosis. Chimeras of the invention may act as agonists to stimulate receptor activation and associated changes in cell activity, or chimeras may be antagonists which block receptor function.

The invention also provides for pharmaceutical compositions comprising a therapeutically effective amount of the nucleic acids of the invention together with a pharmaceutically acceptable adjuvant. Nucleic acid compositions will be suitable for delivery to cells and tissues as part of an anti-sense or gene therapy regimen.

The following examples are offered to more fully illustrate the invention, but are not construed as limiting the scope thereof.

### EXAMPLE 1

Construction and Expression of TNFbp/OPG Fusion Proteins

The TNFbp/OPG[196-401] chimeric gene was prepared in a two step PCR process. A first round of PCR was designed to produce overlapping PCR products from each gene. The templates used were plasmids p2302, containing the gene encoding TNFbp 4.0 (FIG. 4) fused to the Fc region of human IgGl, and plasmid pRcCMV-human OPG (ATCC accession no. 69969), containing the gene for human OPG. The PCR products were gel purified and used as a template to create the chimeric gene. Primers used for the PCR reactions are as follows: 1275-51 (containing a 5′ XbaI site, consensus Kozak and the start of the hTNFbp gene) and 1368-82 (containing a portion of OPG cDNA, an AgeI site and the 3′ end of the human TNFbp 4.0 sequence) were used to amplify the TNFbp gene from p2302; 1368-83 (containing the 3′ end of TNFbp, an AgeI site and the 5′ end of the hOPG C-terminal domain) and 1295-27 (containing a SalI site and the 3′ end of the OPG cDNA) were used to amplify the OPG[196-401] gene from pRcCMV-human OPG. A second PCR reaction used primers 1275-51 and 1295-27 to generate the chimeric gene.

The PCR product was cut with XbaI/SalI and subcloned into the pDSRa2 expression vector to give plasmid p389-1. The expression cassette contains a SV40 early promoter driving the expression of the chimeric gene and also includes an SV40 late intron, an HTLV translation enhancing signal and an α2-FSH polyadenylation signal (DeClerck, et al. J. Biol. Chem. 266, 3893-3899 (1991)). The pDSRα2 vector also contains a DHFR cassette for selection in CHO d-cells.

| | |
|---|---|
| Primer Sequences: | |
| 1275-51: | |
| 5′-CGC TCTAGA CCACC ATG GGC CTC TCC ACC GTG-3′ | (SEQ ID NO._) |
| XbaI Kozak M G L S T V | |
| 1368-82: | |
| 5′-ACACAGGGTAACATCTAT ACCGGT GGTGCCTGAGTCCTCAG-3′ | (SEQ ID NO._) |
| hOPG C-terminus AgeI hTNFbp | |
| 1368-83: | |
| 5′-CTGAGGACTCAGGCACC ACCGGT ATAGATGTTACCCTGTG-3′ | (SEQ ID NO._) |
| E D S G T T G I D V T L | |
| TNFbp AgeI hOPG C-terminus | |
| 1295-27: | |
| 5′-CCTCT GTCGAC TA TTA TAA GCA GCTTATTTTCACGGATTG-3′ | (SEQ ID NO._) |
| SalI * * L C.... OPG--> | |

Other constructs with truncated OPG dimerization doamins were created as follows:

The primer pair for OPG[194-401] was

| | |
|---|---|
| 1295-27 and 1428-89: | |
| TCA ACCGGT AAA TGT GGA ATA GAT GTT AC | (SEQ ID NO:_) |
| AgeI K C G I D V T | |

The primer pair for OPG[217-401] was
1295-27 and 1388-50.

| | |
|---|---|
| 1388-50: | |
| GTTT ACCGGT CCT AAC TGG CTT AGT GTC | (SEQ ID NO:_) |
| AgeI P N W L S V | |

The primer pair for OPG[248-401] was
1295-27 and 1388-51.

| | |
|---|---|
| 1388-51: | |
| AGC ACCGGT GAA CAG ACT TTC GAG CTG | (SEQ ID NO:_) |
| AgeI E Q T F Q L | |

The primer pair for OPG[304-401] was
1295-27 and 1388-52.

| | |
|---|---|
| 1388-52: | |
| GGAA ACCGGT CCG GGA AAG AAA GTG GG | (SEQ ID NO:_) |
| AgeI P G K K V G | |

The corresponding TNFbp/OPG fusion was constructed by excising the AgeI/SalI OPG fragment from p389-1 and replacing it with AgeI/SalI digested OPG PCR products from the above reactions. The amino acid sequences encoded by the above TNFbp/OPG contructs are shown in FIG. 4.

Transient transfections were performed in COS-7 cells by electroporation. Ten μg of plasmid DNA was electroporated into 2×10⁶ cells in 0.8 mls of DMEM. The electroporations were done in 0.4 cm cuvettes at 1.6 kV, 25 mF and 200 ohms. The electroporated cells were plated in 10-cm dishes in DMEM containing 10% FBS, 1× glutamine/penicillin/streptomycin, 1× non-essential amino acids, 1× Na-pyruvate. The following day the media was changed to media containing only 1% FBS. After an additional 72 hours, the conditioned media was harvested and 17 μl was electrophoresed on a 12% denaturing, non-reducing gel. These gels were blotted and analyzed by western blots for the presence of monomer and covalently-linked dimers. The primary antibody was anti-TNFbp (R&D systems, AB-225-PB) at a 1:1000 dilution and the secondary antibody was HRP, rabbit anti-goat,(Pierce) at a 1:1000 dilution.

Stable transfections were done in CHO d-cells by calcium phosphate precipitation (DeClerck et al., suira). The transfection was performed as described except that 20 μg of PvuI linearized plasmid was used with 10 μg of herring sperm carrier DNA and 10 μl of calcium phosphate maximizer (Clontech) to transfect to a 10-cm dish containing approximately 5×10⁵ cells. After 2 weeks in HT-selection, colonies were ring-cloned and expanded into 24-well plates. Once confluent, two day serum-free conditioned media (SFCM) was prepared and analyzed for the expression of TNFbp/OPG fusion protein by western blot. High expressing clones were expanded and grown in roller bottles for 7d SFCM harvests. The results are shown in FIG. 5.

### EXAMPLE 2

Biological Activity of TNFbp/OPG Chimeric Proteins

WEHI Cytotoxicity Assay

The WEHI assay is an in vitro cell proliferation assay (Edwards et al. Endocrinology 128,989-996 (1991)). The cell lines are sensitive to TNF-α (i.e., TNF-α is cytotoxic). In the presence of a TNF-α inhibitor, the cells were protected from the cytotoxic effect and thus were able to proliferate.

TNF-sensitive WEHI 164 clone 13 cells are suspended at a concentration of 20×10⁴ cells/ml in RPMI (Gibco, Grand Island, N.Y.) medium supplemented with 5% Fetal Calf Serum (Hyclone) and penicillin 50U/ml:streptomycin 50 mg/ml. One hundred microliters of this cell suspension are placed in each well of flat-bottomed 96-cell microtiter plates, and the cells are allowed to adhere for 4-6 hours at 37° C. in 7% CO₂. Medium is then aspirated, and 0.60 mg/ml actinomycin-D (Sigma Chemical Co., St. Louis, Mo.) is added to each well. A standard curve using serial dilutions at 0, 0.001 0.01, 0.1, 1, 10, 100 U/ml recombinant human TNF is run with each assay. Serially diluted 10-fold concentrations of TNFbp/OPG chimeras from serum-free conditioned medium are further diluted in RPMI-1640 medium containing 5% FBS and then added to duplicate wells (50 μl/well) containing adherent WEHI 164 cells after the addition of recombinant mouse TNF-α. WEHI-164 clone 13 cells are incubated for 18 hours at 37° C. in 5% CO₂. Maximal killing is determined by adding 0.02% Triton X-100 (TX-100) to test wells. After incubation, 70 μl medium are aspirated, and 50 μl of a 1 mg/mL solution of the organic dye MTT tetrazolium (3-[4,5-dimethylthiozol-2-yl]2,5-diphenyl tetrazolium bromide; Sigma) is added, and cells are incubated for an additional 4-6 hours. All supernatants are then removed, and 50 μl DMF/SDS solution (20% SDS, and 50% N,N dimethylformamide, pH 4.7) is added to each well. The DMF/SDS solution is pipetted up and down several times until all MTT crystals are dissolved, and cells were incubated for an additional 2-22 hours. The absorbances (abs) are read on a Vmax reader at 570-650. The percent specific cytotoxicity is calculated from optical densities using the formula: % specific cytotoxicity=100% X [abs(cells+medium)−abs(cells+sample)]/abs(cells+medium)−abs(cells +TX-100)]. The number of units of TNF in each sample is determined using the percent specific cytotoxicities of the murine standards.

L929 Cytotoxicity Assay

The L929 cytotoxicity assay is an in vitro cell proliferation assay (Parmely et al. J. Immunol. 151, 389-396 (1993), the disclosure of which is hereby incorporated by reference) which also assesses the cytotoxicity of TNF-α-sensitive killing. The cell lines are sensitive to TNF-α (i.e.; TNF-α is cytotoxic). In the presence of a TNF-α inhibitor, the cells are protected from the cytotoxic effect and thus survive and are able to proliferate.

The L929 cell line was obtained from the American Type Culture Collection (catalog number ATCC CCL 1 NCTC clone 929), as described previously by Parmely et. al. (1993), supra. L929 cells were grown in tissue culture flasks in Dulbecco's MEM with 10% fetal calf serum (FCS) to 80% confluence. Cells were trypsinized and seeded at 8,000-10,000 cells/well in 100 ml into Falcon #3072 96 well plates and incubated for 20 to 40 hours at 37 ° C. in 5% CO₂. Samples of TNFbp/Fc or TNFbp/OPG[194-401] polypeptides were serially diluted in medium and added in triplicate followed by addition of TNFα to reach a final concentration of 0.5 mg/ml. The cultures were incubated at 37° C. overnight and cell density was measured by crystal violet. Medium was removed by inverting the 96 well plates. Cells were fixed in 100 μl 100% methanol for 2 minutes. After removal of methanol the plates were allowed to dry for 10 minutes. 100 μl of 0.10% crystal violet stain in 20% methanol was added and plates were Ststained for 10 minutes at room temperature. Excess stain was removed by inverting plates. Plates were washed by dipping three times in ice-cold distilled water and excess water was removed from the wells by gently blotting plates on a tissue. 100 μl of 100% methanol was added to stained cells and optical density was measured at 595 nm. Media control reactions contained L929 cells and medium alone, and TNF control reactions contained L929 cells with 0.5 ng/ml TNFα.

The activity in this assay of TNFbp/OPG fusions constructed as described in Example 1 is shown in FIG. 6.

While the present invention has been described in terms of the preferred embodiments, it is understood that variations and modifications will occur to those skilled in the art. Therefore, it is intended that the appended claims cover all such equivalent variations which come within the scope of the invention as claimed.

What is claimed is:

1. A chimeric polypeptide comprising an amino acid sequence of an osteoprotegerin dimerization domain fused to a heterologous amino acid sequence.

2. The polypeptide of claim 1 wherein the heterologous amino acid sequence and the osteoprotegerin dimerization domain are human.

3. The polypeptide of claim 1 wherein the heterologous amino acid sequence and the osteoprotegerin dimerization domain are from different species.

4. The polypeptide of claim 1 covalently associated with one or more chimeric polypeptides which result in a mulitmeric polypeptide complex.

5. The polypeptide of claim 4 wherein the complex is a dimer.

6. The polypeptide of claim 1 wherein the heterologous amino acid sequence is-a membrane-bound receptor lacking functional membrane associated amino acid sequences.

7. The polypeptide of claim 6 wherein the receptor is selected from the group consisting of receptor tryrosine kinases, cytokine receptors, seven transmembrane domain receptors, and cell adhesion receptors.

8. The polypeptide of claim 1 wherein the heterologous amino acid sequence is selected from members of the tumor necrosis factor-like receptor family consisting of TNFR-1, TNFR-2, TNFrp, NGFR, FasB, CD40, OX40, CD27, CD30, and 4-1BB.

9. The polypeptide of claim 8 wherein the heterologous sequence comprises TNFR-1 lacking functional membrane-associated sequences.

10. The polypeptide of claim 9 wherein the heterologous sequence is a 30 kDa TNF inhibitor, a 40 kDa TNF inhibitor, or an analog thereof.

11. The polypeptide of claim 1 wherein the carboxy terminus of the heterologous sequence is fused to the amino terminus of the OPG dimerization domain.

12. The polypeptide of claim 1 wherein the amino terminus of the heterologous sequence is fused to the carboxy terminus of the OPG dimerization domain.

13. The polypeptide of claim 1 wherein one or more amino acids are inserted between the heterologous sequence and the OPG dimerization domain.

14. A multimeric polypeptide comprising covalently associated monomers of OPG chimeric polypeptides.

15. The multimeric polypeptide of claim 14 which is a dimer.

16. An isolated nucleic acid sequence encoding the polypeptide of claim 1.

17. An expression vector comprising the nucleic acid sequence of claim 16.

18. A host cell transformed or transfected with the expression vector of claim 17 in a manner allowing expression of the nucleic acid.

19. A pharmaceutical composition comprising the polypeptide of any of claims 1 to 15.
